(19) United States
(12) Patent Application Publication
(10) Pub. No.: US 2009/0142289 A1
(43) Pub. Date: Jun. 4, 2009

(54) COMPOSITION CONTAINING A HARD WAX AND A PASTY COMPOUND, AND PROCESS FOR THE CARE OR MAKING UP OF THE EYELASHES

(75) Inventors: Stephane ARDITTY, Ballainvilliers (FR); Sandrine Rimbert, Antony (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/198,185

(22) Filed: Aug. 26, 2008

Related U.S. Application Data
(60) Provisional application No. 60/987,419, filed on Nov. 13, 2007.
(60) Provisional application No. 60/987,422, filed on Nov. 13, 2007.

(30) Foreign Application Priority Data
Aug. 28, 2007 (FR) 07 57220
Aug. 28, 2007 (FR) 07 57221

(51) Int. Cl. A61K 8/92; A61Q 1/10

(52) U.S. Cl. 424/70.7

(57) ABSTRACT

Composition containing an aqueous phase, at least 10% by weight of at least one hard wax, relative to the total weight of the composition, and at least one pasty compound. Also described is a composition containing an aqueous phase, at least one hard wax, at least one pasty compound, and possibly at least one oil, the hard wax(es)/(pasty compound(s)+possible oil(s)) weight ratio ranging from 2 to 8. The uses thereof for the care or the making up of keratinous fibres and in particular the eyelashes are also described.

### REFERENCE TO PRIOR APPLICATIONS

This application claims priority to U.S. provisional applications 60/987,419 and 60/987,422 both filed Nov. 13, 2007, and to French patent applications 0757220 and 0757221, both filed Aug. 28, 2007, all incorporated herein by reference.

### FIELD OF THE INVENTION

The invention relates to a composition comprising an aqueous phase, at least one hard wax and at least one pasty compound, a process for making up or care of keratinous fibres such as the eyelashes, the eyebrows and the hair, and more particularly the eye-lashes, using the composition, and the use thereof for making up of the fibres, and more particularly as mascara.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In the cosmetics field, compositions for coating the eyelashes, such as mascaras, are generally make-up compositions, compositions for application onto make-up (also referred to as “top-coat”) or even compositions for the cosmetic care of the eyelashes.

The mascaras are in particular prepared on the basis of two types of formulation: aqueous mascaras referred to as cream mascaras, in the form of a dispersion of waxes in water, and anhydrous or low-water-content mascaras, referred to as water-resistant mascaras (referred to as “waterproof”), in the form of dispersions of waxes in organic solvents.

The present application more specifically relates to the aqueous mascaras field.

The application of mascara makes it possible to increase the volume of the eyelashes and consequently to increase the intensity of the gaze. For this, there are many thickening or filling mascaras, imparting volume. This filling effect is generally obtained by depositing a maximum of solid substances onto the eyelashes.

It is in particular through the qualitative and quantitative choice of the solid particles, in particular the waxes, that the application properties sought for make-up compositions, such as for example their fluidity or consistency, their covering power or their thickening power (also called filling or make-up power) can be adjusted.

In order to adjust the consistency and the cosmeticity of these compositions, so-called “soft” waxes, such as beeswax or paraffin wax, are used in combination with so-called “hard” waxes in order to obtain a mascara exhibiting a high solids content and a medium to high consistency, these characteristics being necessary for a mascara to be filling.

In addition, the aqueous mascaras mainly contain a surfactant system, for example based on triethanolamine stearate, which makes it possible to obtain a stable dispersion of particles of wax agglomerated in an aqueous phase. This system plays an important part in the obtaining of such a dispersion, in particular at the interface in the interactions between particles of wax.

However, the mascaras described above have the disadvantages of having a dry nature on application and a consistency which is not easy to work, as well as the formation of a granular deposit when the quantity of solid substances is too high.

The use of “soft” waxes in these compositions makes it possible to obtain somewhat less granular deposits, but imparts an adhesive nature to the final deposit, which leads to adhesion of the made-up eyelashes when they are simply rubbed with a finger.

There is thus a need to develop a cosmetic composition, in particular for making up the eyelashes, making it possible to obtain a smooth and homogeneous deposit on the eyelashes and a filling effect, while exhibiting a consistency that is easy to work after application, and does not dry too rapidly.

### SUMMARY OF THE INVENTION

The inventors have surprisingly discovered that it is possible to formulate aqueous-phase compositions that overcome the disadvantages described above, by using in these compositions at least 10% by weight of at least one hard wax, relative to the total weight of the composition, and at least one pasty compound.

The inventors have also found that the disadvantages described above are overcome with a composition comprising an aqueous phase, at least one hard wax, at least one pasty compound and optionally at least one oil, the hard wax(es)/(pasty compound(s)+optional oil(s)) weight ratio ranging from 2 to 8.

### DETAILED DESCRIPTION OF THE PREFERRED EMBODIMENTS

One subject of the invention is thus compositions, in particular cosmetic compositions for the making up or care of keratinous fibres, comprising an aqueous phase, at least 10% by weight of at least one hard wax, relative to the total weight of the composition, and at least one pasty compound.

Another subject of the invention are compositions, in particular for the making up or care of keratinous fibres, comprising an aqueous phase, at least one hard wax, at least one pasty compound, and optionally (sometimes the term “possibly” is used herein for optionally) at least one oil, the hard wax(es)/(pasty compound(s)+optional oil(s)) weight ratio ranging from 2 to 8, including 2, 3, 4, 5, 6, 7 and 8 and all values and subranges in-between 2-8 as if explicitly written out.

Other subjects of the invention include processes for the making up or care of keratinous fibres, in particular the eyelashes, the eyebrows and the hair, and more particularly the eyelashes, using the compositions according to the invention.

It also relates to the use of the compositions according to the invention as mascaras.

Other subject matters and characteristics, aspects and advantages of the invention will appear still more clearly in the reading of the description and the example that follows.

In one embodiment the composition according to the invention comprises an aqueous phase, at least 10% by weight of at least one hard wax, relative to the total weight of the composition, and at least one pasty compound.

This composition can further contain at least one oil. The hard wax(es)/(pasty compound(s)+possible oil(s)) weight ratio preferably then ranges from 2 to 8, including 2, 3, 4, 5, 6, 7 and 8 and all values and subranges in-between 2-8 as if explicitly written out.

Another embodiment of the invention is a composition comprising an aqueous phase, at least one hard wax, at least one pasty compound, and possibly at least one oil, the hard wax(es)/(pasty compound(s)+possible oil(s)) weight ratio ranging from 2 to 8, including 2, 3, 4, 5, 6, 7 and 8 and all values and subranges in-between 2-8 as if explicitly written out.

In another embodiment, the hard wax(es)/(pasty compound(s)+possible oil(s)) weight ratio ranges from 2 to 6, better still from 2.5 to 6, and still more preferably from 3 to 5.

The compositions according to the invention preferably contains a total quantity of pasty compound(s) and oil(s) greater than 1% by weight, better still ranging from 1 to 15% by weight, preferably greater than 2% by weight, and for example ranging from 2 to 10% by weight, and still more preferably ranging from 3 to 10% by weight relative to the total weight of the composition.

The aqueous phase contained in the compositions according to the invention can form the continuous phase of the compositions.

A composition with a continuous aqueous phase is understood to mean a composition exhibiting a conductivity, measured at 25° C., greater than or equal to 23 μS/cm (microSiemens/cm), the conductivity being measured, for example, by means of a Mettler Toledo MPC227 conductimeter and an Inlab730 conductivity measurement cell. The measurement cell is immersed in the composition, so as to eliminate air bubbles capable of forming between the 2 electrodes of the cell. The conductivity reading is taken when the conductimeter value has stabilised. A mean is taken over at least 3 consecutive measurements.

The aqueous phase contains water and possibly at least one water-soluble solvent.

In the sense of the present invention, “water-soluble solvent” is understood to mean a compound which is liquid at ambient temperature and miscible with water (miscibility with water greater than 50% by weight at 25° C. and atmospheric pressure).

The water-soluble solvents utilisable in the compositions according to the invention can moreover be volatile.

Among the water-soluble solvents that can be used in the compositions according to the invention, lower monohydric alcohols having from 1 to 5 carbon atoms such as ethanol and isopropanol, alkylene glycols having from 2 to 8 carbon atoms such as ethylene glycol, propylene glycol, 1,3-butylene glycol and dipropylene glycol, C₃-C₄ ketones and C₂-C₄ aldehydes can in particular be cited.

The aqueous phase is generally present in the compositions according to the invention at a level ranging from 1 to 95% by weight, relative to the total weight of the composition, preferably ranging from 3 to 80% by weight, and more preferably ranging from 5 to 60% by weight.

In the sense of the present invention, wax is understood to mean a lipophilic compound, solid at ambient temperature (25° C.), with a reversible solid/liquid change of state, having a melting point greater than or equal to 30° C., and which may be up to 120° C. In particular, the waxes have a melting point greater than 30° C. and better greater than 45° C.

In the sense of the invention, the melting point corresponds to the temperature of the most endothermic peak observed in thermal analysis (differential calorimetric analysis or DSC) such as is described in the standard ISO 11357-3; 1999. The melting point of the wax can be measured by means of a differential scanning calorimeter (DSC), for example the calorimeter sold under the name “MDSC 2920” by the company TA Instruments.

The measurement protocol is as follows:

A sample of 5 mg of wax placed in a crucible is subjected to a first temperature rise passing from −20° C. to 100° C., at the heating rate of 10° C./minute, then is cooled from 100° C. to −20° C. at a cooling rate of 10° C./minute and finally subjected to a second temperature rise passing from −20° C. to 100° C. at a heating rate of 5° C./minute. During the second temperature rise, the variation in the difference in the power absorbed by the empty crucible and by the crucible containing the wax sample as a function of the temperature is measured. The melting point of the compound is the temperature value corresponding to the summit of the peak in the curve representing the variation in the difference in the power absorbed as a function of the temperature.

In the sense of the present invention, a hard wax is understood to mean a wax exhibiting a hardness greater than 5 MPa, in particular ranging from 5 to 30 MPa, preferably greater than 6 MPa, better still ranging from 6 to 25 MPa, at 20° C.

The hardness of the wax is determined by measurement of the compression force measured at 20° C. by means of the texturometer sold under the name TA-XT2 by the company RHEO, equipped with a stainless steel cylinder with a diameter of 2 mm moving at the measurement speed of 0.1 mm/s, and penetrating into the wax to a penetration depth of 0.3 mm.

The measurement protocol is as follows: the wax is melted at a temperature equal to the melting point of the wax+10° C. The melted wax is poured into a container 25 mm in diameter and 20 mm deep. The wax is recrystallised at ambient temperature (25° C.) for 24 hours so that the surface of the wax is flat and smooth, then the wax is kept for at least 1 hour at 20° C. before performing the measurement of the hardness or the adhesivity.

The mobile element of the texturometer is moved at the speed of 0.1 mm/s, then penetrates into the wax to a penetration depth of 0.3 mm. When the mobile element has penetrated into the wax to the depth of 0.3 mm, the mobile element is kept immobile for 1 second (corresponding to the relaxation time) and is then withdrawn at the speed of 0.5 mm/s.

The value of the hardness is the maximal compression force measured, divided by the area of the texturometer cylinder in contact with the wax.

The waxes can be hydrocarbon, fluorinated and/or silicone, and be of plant, mineral, animal and/or synthetic origin.

As examples of hard wax, Carnauba wax, candelilla wax, BIS-PEG-12 DIMETHICONE CANDELILLATE wax such as for example the Siliconyl Candelilla Wax marketed by the company KOSTER KEUNEN, hydrogenated Jojoba wax such as for example that marketed by the company DESERT WHALE, hydrogenated palm oil such as that marketed by the company SIO, rice bran wax, Sumac wax, ceresin waxes, laurel wax, Chinese insect wax, Shellac wax, hydrogenated olive oil such as Waxolive from the company SOLIANCE, waxes obtained by hydrogenation of olive oil esterified with C12 to C18 chain fatty alcohols such as those sold by the company SOPHIM under the brand names Phytowax Olive 12L44, 14L48, 16L55 and 18L57, waxes obtained by hydrogenation of castor oil esterified with cetyl or behenyl alcohol such as for example those which are sold under the names Phytowax Ricin 16 L 64 and Phytowax Ricin 22 L 73 by the company SOPHIM, hydrogenated Cameline wax, Ouricury wax, Montan wax, ozokerite waxes such as for example Wax SP 1020 P marketed by the company Strahl & Pitsch, microcrystalline waxes such as for example that sold under the brand name Microwax HW by the company PARAMELT, triglycerides of lauric, palmitic, cetylic and stearic acids (INCI name: hydrogenated coco glycerides) such as for example that sold under the brand name Softisan 100 by the company SASOL, polymethylene waxes such as for example that sold under the brand name Cirebelle 303 by the company SASOL, polyethylene waxes such as for example those sold under the brand names Performalene 400 polyethylene, Performalene 655 polyethylene and Performalene 500-L polyethylene by the company New Phase Technologies, alcohol-polyethylene waxes such as for example that marketed under the name Performacol 425 Alcohol by the company BARECO, the 95/5 ethylene/acrylic acid copolymer sold under the brand name AC 540 wax by the company Honeywell, hydroxyoctacosanyl hydroxy-stearate such as for example that sold under the brand name Elfacos C 26 by the company AKZO, octacosanyl stearate such as for example that marketed under the name Kester Wax K 82H by the company KOSTER KEUNEN, stearyl stearate such as for example that marketed under the name Liponate SS by the company LIPO CHEMICALS, pentaerythritol distearate such as for example that marketed under the name Cutina PES by the company COGNIS, the mixture of dibehenyl adipate, dioctadecyl adipate and di-eicosanyl adipate (INCI name C₁₈₋₂₂ dialkyl adipate), the mixture of dilauryl adipate and ditetradecyl adipate (INCI name: C₁₂₋₁₄ dialkyl adipate), the mixture of dioctadecyl sebacate, didocosyl sebacate and dieicosyl sebacate (INCI name: C₁₈₋₂₂ dialkyl sebacate) and the mixture of dioctadecyl octadecanedioate, didocosyl octanedioate and dieicosyl octanedioate (INCI name: C₁₈₋₂₂ dialkyl octanedioate) such as for example those marketed by the company COGNIS, pentaerythrityl tetrastearate such as for example Liponate PS-4 from the company Lipo Chemicals, tetracontanyl stearate such as for example Kester Wax K76H from the company KOSTER KEUNEN, stearyl benzoate such as for example Finsolv 116 from the company FINETEX, behenyl fumarate such as for example Marrix 222 from the company AKZO BERNEL, di-(trimethylol-1,1,1-propane) tetrastearate such as for example that which is offered under the name “HEST 2T-4S” by the company HETERENE, didotriacontanyl distearate such as for example Kester Wax K82D from the company KOSTER KEUNEN, polyethylene glycol montanate with 4 ethylene oxide units (PEG-4) such as for example that which is sold under the brand name Clariant Licowax KST1, hexanediol disalicylate such as for example Betawax RX-13750 marketed by the company CP Hall, dipentaerythritol hexastearate such as for example that which is sold under the brand name Hest 2P-6S by the company HETERENE, ditrimethylolpropane tetrabehenate such as for example that which is sold under the brand name Hest 2T-4B by the company HETERENE, Jojoba esters such as for example that which is sold under the brand name Floraester HIP by the company FLORATECH, mixtures of linear (C20-40) carboxylic acid/saturated hydrocarbons (INCI name: C20-40 acid polyethylene) such as for example Performacid 350 acid from the company NEW PHASE TECHNOLOGIES, synthetic wax of the Fischer-Tropsch type such as that marketed under the name Rosswax 100 by the company ROSS, cetyl alcohol, stearyl alcohol, behenyl alcohol, dioctadecyl carbonate such as for example Cutina KE 3737, saccharose polybehenate such as for example Crodaderm B from the company CRODA, and mixtures thereof, can in particular be cited.

The waxes cited above can also be used in the form of commercially available mixtures, for example, under the names KOSTER KPC-56 (mixture of 87.5% by weight of cetyl stearate, 7.5% by weight of behenyl alcohol and 5% by weight of palm kernel glycerides), KPC-60 (mixture of 87.5% by weight of stearyl stearate, 7.5% by weight of behenyl alcohol 5% by weight of palm kernel glycerides), KPC-63 (mixture of 87.5% by weight of behenyl stearate, 7.5% by weight of behenyl alcohol and 5% by weight of palm kernel glycerides) and KPC-80 (mixture of 86% by weight of synthetic beeswax, 7.5% of hydrogenated plant oil and 6.5% by weight of behenyl alcohol) from the company KOSTER KEUNEN.

Waxes of plant origin such as carnauba wax, candelilla wax, hydrogenated jojoba wax, sumac wax, waxes obtained by hydrogenation of olive oil esterified with C12 to C18 chain fatty alcohols sold by the company SOPHIM in the Phytowax range (12L44, 14L48, 16L55 and 18L57), rice bran wax, cetyl, stearyl and behenyl alcohols, laurel wax and Ouricury wax are preferably used.

The hard wax(es) are preferably present in a quantity of at least 12% by weight, better still from 12 to 30% by weight, still more preferably at least 14% by weight, and above all from 14 to 25% by weight relative to the total weight of the composition.

In the sense of the present invention, “pasty compound” is understood to mean a lipophilic fatty compound with a reversible solid/liquid change of state and containing a liquid fraction and a solid fraction at the temperature of 23° C.

A pasty compound is in the form of a liquid fraction and a solid fraction at the temperature of 23° C. In other words, the starting melting point of the pasty compound is lower than 23° C. The liquid fraction of the pasty compound, measured at 23° C., represents from 20 to 97% by weight of the pasty compound. At 23° C., this liquid fraction more preferably represents from 25 to 85%, and better from 30 to 60% by weight of the pasty compound.

The liquid fraction by weight of the pasty compound at 23° C. equals the ratio of the melting enthalpy consumed at 23° C. to the melting enthalpy of the pasty compound.

The melting enthalpy consumed at 23° C. is the quantity of energy absorbed by the sample in order to pass from the solid state to the state it exhibits at 23° C., consisting of a liquid fraction and a solid fraction.

The melting enthalpy of the pasty compound is the enthalpy consumed by the compound in order to pass from the solid state to the liquid state. The pasty compound is said to be in the solid state when the entirety of its mass is in solid form. The pasty compound is said to be in the liquid state when the entirety of its mass is in liquid form.

The melting enthalpy of the pasty compound equals the area under curve of the thermogram obtained by means of a differential scanning calorimeter (D. S. C.), such as the calorimeter sold under the name MDSC 2920 by the company TA instruments, with a temperature rise of 5 or 10° C. par minute, in accordance with the standard ISO 11357-3:1999. The melting enthalpy of the pasty compound is the quantity of energy necessary to make the compound pass from the solid state to the liquid state. It is expressed in J/g.

The liquid fraction of the pasty compound, measured at 32° C., preferably represents from 40 to 100% by weight of the pasty compound, better still from 50 to 100% by weight of the pasty compound. When the liquid fraction of the pasty compound measured at 32° C. equals 100%, the temperature of the end of the melting range of the pasty compound is less than or equal to 32° C.

The liquid fraction of the pasty compound, measured at 32° C., equals the ratio of the melting enthalpy consumed at 32° C. to the melting enthalpy of the pasty compound. The melting enthalpy consumed at 32° C. is calculated in the same manner as the melting enthalpy consumed at 23° C.

The pasty compound preferably has a hardness at 20° C. ranging from 0.001 à 0.5 MPa, preferably from 0.002 to 0.4 MPa.

The hardness is measured by a method of penetration of a probe into a sample of compound and in particular by means of a texture analyser (for example the TA-XT2i from Rhéo) equipped with a stainless steel cylinder 2 mm in diameter. The hardness measurement is carried out at 20° C. at the centre of 5 samples. The cylinder is introduced into each sample, the penetration depth being 0.3 mm. The value stated for the hardness is that of the maximum peak.

The pasty compound can be selected from synthetic compounds and compounds of plant origin. A pasty compound can be obtained by synthesis from starting materials of plant origin.

The pasty compound is advantageously selected from:
lanolin and derivatives thereof such as lanolin alcohol, ethoxylated lanolins, acetylated lanolin, esters of lanolin such as isopropyl lanolate, and propoxylated lanolins,
polymeric or non-polymeric silicone compounds such as high-molecular-weight polydimethylsiloxanes, polydimethylsiloxanes with side chains of the alkyl or alkoxy type having from 8 to 24 carbon atoms, in particular the stearyl dimethicones,
polymeric or non-polymeric fluorinated compounds,
vinyl polymers, in particular
   homopolymers of olefins,
   copolymers of olefins,
   homopolymers and copolymers of hydrogenated dienes,
   linear or branched, homo- or copolymeric oligomers of alkyl (meth)acrylates preferably having a C₈-C₃₀ alkyl group,
   homo- or copolymeric oligomers of vinyl esters having C₈-C₃₀ alkyl groups,
   homo- or copolymeric oligomers of vinyl ethers having C₈-C₃₀ alkyl groups,
liposoluble polyethers resulting from polyetherification between one or more C₂-C₁₀₀, preferably C₂-C₅₀, diols,
esters and polyesters,
and mixtures thereof.

The pasty compound can be a polymer, in particular a hydrocarbon polymer.

A preferred silicone and fluorinated pasty compound is poly-methyl-trifluoropropyl-methylalkyl-dimethylsiloxane, manufactured under the name X22-1088 by SHIN ETSU.

When the pasty compound is a silicone and/or fluorinated polymer, the composition advantageously contains a compatiblising agent such as short chain esters such as isodecyl neopentanoate.

Among the liposoluble polyethers, copolymers of ethylene oxide and/or propylene oxide with C₆-C₃₀ alkylene oxides can in particular be cited. Preferably, the ratio by weight of the ethylene oxide and/or propylene oxide to the alkylene oxides in the copolymer is from 5:95 to 70:30. In this family, block copolymers containing blocks of C₆-C₃₀ alkylene oxide having a molecular weight ranging from 1,000 to 10,000, for example a polyoxyethylene/polydodecylene glycol block copolymer such as ethers of dodecanediol (22 mol) and polyethylene glycol (45 ethylene oxide or EO units) marketed under the trademark ELFACOS ST9 by Akzo Nobel will in particular be cited.

Among the esters, the following are particularly preferred:
esters of an oligomeric glycerol, in particular esters of diglycerol, in particular condensation products of adipic acid and glycerol, for which some of the hydroxyl groups of the glycerols have reacted with a mixture of fatty acids such as stearic acid, capric acid, stearic acid, isostearic acid and 12-hydroxystearic acid, such as in particular those marketed under the trademark Softisan 649 by the company Sasol;
   esters of phytosterol;
   esters of pentaerythritol;
   esters formed from:
      at least one C₁₆₋₄₀ alcohol, at least one of the alcohols being a Guerbet alcohol and
      a dimeric diacid formed from at least one unsaturated C18-40 fatty acid,
   such as the ester of tall oil fatty acids dimer containing 36 carbon atoms and a mixture of i) Guerbet alcohols containing 32 carbon atoms and ii) behenyl alcohol; the ester of linoleic acid dimer and a mixture of two Guerbet alcohols 2-tetradecyl-octadecanol (32 carbon atoms) and 2-hexadecyl-eicosanol (36 carbon atoms);
   non-crosslinked polyesters resulting from polycondensation between a linear or branched C₄-C₅₀ dicarboxylic acid or poly-carboxylic acid and a C₂-C₅₀ diol or polyol;
   polyesters which result from esterification between a polycarboxylic acid and an ester of a hydroxylated aliphatic carboxylic acid such as Risocast DA-L and Risocast DA-H marketed by the Japanese company KOKYU ALCOHOL KOGYO, which are esters resulting from the esterification reaction of hydrogenated castor oil with dilinoleic acid or isostearic acid; and
   aliphatic ester esters resulting from esterification between a hydroxylated aliphatic carboxylic acid and an aliphatic carboxylic acid, for example that sold under the brand name Salacos HClS (V)-L by the company Nishing Oil.

A Guerbet alcohol is the reaction product from the Guerbet reaction, well known to the person skilled in the art. This is a reaction transforming a primary aliphatic alcohol into the β-alkylated dimeric alcohol therefrom with loss of one equivalent of water.

The aliphatic carboxylic acids described above generally contain from 4 to 30 and preferably from 8 to 30 carbon atoms. They are preferably selected from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, isostearic acid, nonadecanoic acid, eicosanoic acid, isoarachidic acid, octyldodecanoic acid, heneicosanoic acid, docosanoic acid and mixtures thereof.

The aliphatic carboxylic acids are preferably branched.

The hydroxylated aliphatic carboxylic acid esters are advantageously derived from a hydroxylated aliphatic carboxylic acid containing from 2 to 40 carbon atoms, preferably from 10 to 34 carbon atoms and better from 12 to 28 carbon atoms, and from 1 to 20 hydroxyl groups, preferably from 1 to 10 hydroxyl groups and better from 1 to 6 hydroxyl groups. The hydroxylated aliphatic carboxylic acid esters are in particular selected from:

a) partial or total esters of linear, saturated monohydroxylated aliphatic monocarboxylic acids;

b) partial or total esters of unsaturated monohydroxylated aliphatic monocarboxylic acids;

c) partial or total esters of saturated monohydroxylated aliphatic polycarboxylic acids;

d) partial or total esters of saturated polyhydroxylated aliphatic polycarboxylic acids;

e) partial or total esters of C₂ to C₁₆ aliphatic polyols which have reacted with a mono- or polyhydroxylated aliphatic mono- or polycarboxylic acid,

f) and mixtures thereof.

The aliphatic esters of ester are advantageously selected from:
the ester resulting from esterification reaction of hydrogenated castor oil with isostearic acid in the proportions 1 to 1 (1/1), which is called hydrogenated castor oil isostearate,
the ester resulting from the esterification reaction of hydrogenated castor oil with isostearic acid in the proportions 1 to 2 (1/2), which is called hydrogenated castor oil diisostearate,
the ester resulting from the esterification reaction of hydrogenated castor oil with isostearic acid in the proportions 1 to 3 (1/3), which is called hydrogenated castor oil triisostearate,
and mixtures thereof.

Preferably, the pasty compound is selected from compounds of plant origin.

Among these, isomerised jojoba oil such as the trans isomerised partially hydrogenated jojoba oil manufactured or marketed by the company Desert Whale under the trade name Iso-Jojoba-50®, orange wax such as for example that marketed under the name Orange Peel Wax by the company Koster Keunen, shea butter, partially hydrogenated olive oil such as for example the compound marketed under the name Beurrolive by the company Soliance, cocoa butter, and mango oil such as for example Lipex 302 from the company Aarhuskarlshamn can in particular be cited.

The pasty compound or compounds are preferably present in a quantity greater than or equal to 1% by weight relative to the total weight of the composition, for example from 1 to 15% by weight, better in a quantity greater than or equal to 2% by weight, for example ranging from 2 to 10% by weight, and still more preferably from 3 to 8% by weight, relative to the total weight of the composition.

The compositions according to the invention advantageously exhibit a dry matter (or dry extract) content greater than or equal to 40% by weight, better greater than or equal to 45% by weight relative to the total weight of the composition.

The dry matter content, that is to say the non-volatile matter content, can be measured in different ways. For example, the oven drying methods, methods by drying by exposure to infrared radiation and chemical methods by Karl Fischer water titration can be cited.

Preferably, the quantity of dry matter, commonly called “dry extract” of the compositions according to the invention, is measured by heating the sample with infrared rays of wavelength 2 μm to 3.5 μm. The substances contained in the compositions which have a high vapour pressure evaporate under the action of this radiation. By measurement of the weight loss of the sample, it is possible to determine the “dry extract” of the composition. These measurements are made by means of an LP16 commercial infrared desiccator from Mettler. This technique is fully described in the documentation for the device provided by Mettler.

The measurement protocol is as follows: about 1 of the composition is spread out on a metal cupel. After introduction into the desiccator, this is subjected to high-temperature storage at 120° C. for one hour. The wet mass of the sample corresponding to the initial mass and the dry mass of the sample corresponding to the mass after exposure to the radiation are measured with a precision balance.

The dry matter content is calculated as follows:

Dry Extract=100×(dry mass/wet mass).

The compositions according to the invention can further contain at least one oil.

“Oil” is understood to mean a non-aqueous substance liquid at ambient temperature and atmospheric pressure. It generally has a melting point lower than 55° C., preferably lower than 50° C., and better still less than or equal to 45° C. The oil can be volatile or non-volatile.

In the sense of the invention, “volatile oil” is understood to mean any non-aqueous medium capable of evaporating in contact with keratinous fibres in less than one hour at ambient temperature and atmospheric pressure. The volatile oils which can be used in the compositions of the invention are volatile cosmetic oils, liquid at ambient temperature, having a non-zero vapour pressure at ambient temperature and atmospheric pressure, ranging from 0.13 Pa to 40,000 Pa (10⁻³ to 300 mm Hg), in particular ranging from 1.3 Pa to 13,000 Pa (0.01 to 100 mm Hg), and more particularly ranging from 1.3 Pa to 1300 Pa (0.01 to 10 mm Hg).

“Non-volatile oil” is understood to mean an oil remaining on the keratinous fibres at ambient temperature and atmospheric pressure for at least several hours and having in particular a vapour pressure less than 10⁻³ mm Hg (0.13 Pa).

The composition according to the invention can contain volatile oils and/or non-volatile oils, or mixtures thereof. They can be hydrocarbon oils, silicone oils, fluorinated oils or mixtures thereof.

“Hydrocarbon oil” is understood to mean an oil containing mainly hydrogen atoms and carbon atoms and possibly atoms of oxygen, nitrogen, sulphur and phosphorus.

The volatile hydrocarbon oils can be selected from the hydrocarbon oils having from 8 to 16 carbon atoms, and in particular branched C₈-C₁₆ alkanes such as the C₈-C₁₆ isoalkanes of petroleum origin (also called isoparaffins) such as isododecane (also called 2,2,4,4,6-pentamethylheptane), isodecane, isohexadecane, and for example the oils sold under the trade name of “Isopars®” or “Permethyls®”; branched C₈-C₁₆ esters such as isohexyl neopentanoate, and mixtures thereof. Other volatile hydrocarbon oils such as petroleum distillates, in particular those sold under the name “Shell Solt®” by the company SHELL, can also be used.

As volatile oils, volatiles silicones such as for example linear or cyclic, volatile silicone oils, in particular those having a viscosity ≦6 centistokes (6.10⁻⁶ m²/s), and having in particular from 3 to 6 silicon atoms, these silicones possibly containing one or more alkyl or alkoxy groups having 1 or 2 carbon atoms can also be used. As volatile silicone oil utilisable in the invention, octamethyl cyclotetrasiloxane, decamethyl cyclopentasiloxane, dodecamethyl cyclohexasiloxane, heptamethyl hexyltrisiloxane, heptamethyloctyl trisiloxane, hexa-methyl disiloxane, octamethyl trisiloxane, decamethyl tetrasiloxane, dodecamethyl pentasiloxane and mixtures thereof can in particular be cited.

Volatile organic solvents, in particular fluorinated ones, such as nonafluoromethoxy butane or perfluoromethylcyclopentane can also be used as the volatile oil.

As non-volatile hydrocarbon oil, the following can in particular be cited:
hydrocarbon oils of plant origin, such as triglycerides made up of esters of linear or branched, saturated or unsaturated, C₄-C₂₄ fatty acids and glycerol. As examples of such oils, wheat germ, sunflower, grape seed, sesame, maize, apricot, castor, shea, avocado, olive, soya, sweet almond, palm, rape, cotton, hazelnut, macadamia, jojoba, lucerne, poppy seed, butternut squash, marrow, blackcurrant, evening primrose, millet, barley, quinoa, rye, safflower, candlenut, passionflower and muscat rose oil, or also the triglycerides of caprylic/capric acids such as those sold by the company Stearineries Dubois or those sold under the names Miglyol 810®, 812® and 818® by the company Dynamit Nobel can in particular be cited;
synthetic ethers having from 10 to 40 carbon atoms;
linear or branched hydrocarbons, of mineral or synthetic origin, such as vaseline, polydecenes, hydrogenated polyisobutene such as parleam, squalane, and mixtures thereof;
synthetic esters such as the oils of formula R₁COOR₂ wherein R₁ represents the residue of a linear or branched fatty acid containing from 1 to 40 carbon atoms and R₂ represents a hydrocarbon chain, in particular branched, containing from 1 to 40 carbon atoms, provided that R₁+R₂ is ≧10. As examples of synthetic esters, Purcellin oil (cetostearyl octanoate), isopropyl myristate, isopropyl palmitate, C₁₂ to C₁₅ alcohol benzoate, hexyl laurate, diisopropyl adipate, isononyl isononanoate, 2-ethylhexyl palmitate, isostearyl isostearate, octanoates, decanoates or ricinoleates of alcohols or polyhydric alcohols such as propylene glycol dioctanoate, hydroxylated esters such as isostearyl lactate, diisostearyl malate and esters of pentaerythritol;
fatty alcohols liquid at ambient temperature and with a branched and/or unsaturated carbon chain having from 12 to 26 carbon atoms such as octyl dodecanol, isostearyl alcohol, oleyl alcohol, 2-hexyldecanol, 2-butyloctanol, and 2-undecylpentadecanol;
saturated or unsaturated C₁₂₋₂₄ fatty acids such as oleic acid, linoleic acid and linolenic acid;
and mixtures thereof can in particular be cited.

The non-volatile silicone oils utilisable in the compositions according to the invention can be non-volatile polydimethylsiloxanes (PDMS), polydimethylsiloxanes containing alkyl or alkoxy groups, lateral and/or at the end of a silicone chain, groups each containing from 2 to 24 carbon atoms, phenylated silicones such as the phenyl trimethicones, phenyl dimethicones, phenyl trimethylsiloxy diphenyl-siloxanes, diphenyl dimethicones, diphenyl methyldiphenyl trisiloxanes and 2-phenylethyl trimethylsiloxysilicates.

The non-volatile fluorinated oils utilisable in the compositions according to the invention are in particular fluorosilicone oils, fluorinated polyethers and fluorinated silicones such as those described in the document EP-A-847752.

When it (they) is (are) present in the composition according to the invention, the content of oil(s) in the composition according to the invention preferably ranges from 0.01 to 30% by weight, in particular from 0.1 to 25% by weight, and better from 0.1 to 20% relative to the total weight of the composition.

The compositions can further contain at least one additional wax exhibiting a hardness less than or equal to 5 MPa, more particularly ranging for example from 0.01 to 5 MPa, at 20° C. The hardness of the additional wax is measured in the same manner as for the hard wax.

The additional wax can be selected from, for example, bees waxes, siliconised beeswax, paraffin waxes, berry wax, lemon wax, the 85/15 ethylene/vinyl acetate copolymer sold under the brand name Cire AC 400, and silicone waxes such as the alkyldimethicone sold under the brand name SF 1642 by the company GE Bayer silicone, and the waxes sold under the brand names K82P-S and K82P-VS by the company Koster Keunen.

Waxes obtained by catalytic hydrogenation of animal or plant oils having linear or branched, C₈-C₃₂ fatty chains such as hydrogenated sunflower oil, hydrogenated castor oil, hydrogenated copra oil and hydrogenated lanolin oil can also be cited.

The silicone waxes and the fluorinate waxes can also be cited.

The additional wax can be a so-called adhesive wax, that is to say one exhibiting adhesivity greater than or equal to 0.1 N·s and a hardness less than or equal to 3.5 MPa.

The adhesive wax used can in particular exhibit adhesivity ranging from 0.1 N·s to 10 N·s, in particular ranging from 0.1 N·s to 5N·s, preferably ranging from 0.2 to 5 N·s and better ranging from 0.3 to 2 N·s.

The adhesivity of the wax is determined by measurement of the change in the compression force as a function of time at 20° C. according to the protocol previously described for the measurement of the hardness.

During the relaxation time of 1 s, the compression force decreases markedly until it becomes zero, then, during the withdrawal of the mobile element, the stretching force becomes negative and then again increases towards the value 0. The adhesivity corresponds to the integral of the curve of the force as a function of the time for the part of the curve corresponding to the negative values of the force. The value of the adhesivity is expressed in N·s.

The adhesive wax which can be used generally has a hardness less than or equal to 3.5 MPa, in particular ranging from 0.01 to 3.5 MPa, in particular ranging from 0.05 to 3 MPa.

As the adhesive wax, a C₁₈-C₃₈ alkyl (hydroxystearoyl)stearate (the alkyl group containing from 18 to 38 carbon atoms) can be used, alone or in a mixture.

Such wax is in particular sold under the names “Kester Wax K 82 P®”, “Hydroxypolyester K 82 P®” and “Kester Wax K 80 P®” by the company KOSTER KEUNEN.

In the present invention, waxes supplied in the form of small particles having a dimension expressed as “effective” mean diameter by volume D[4,3] of the order of 0.5 to 30 micrometres, in particular from 1 to 20 micrometres, and more particularly from 5 to 10 micrometres, referred to below by the expression “micro waxes”, can also be used.

The particle sizes can be measured by different techniques, such as light diffusion techniques (dynamic and static), Coulter counter methods, measurements by sedimentation rate (linked to the size via Stokes' law) and microscopy. These techniques make it possible to measure a particle diameter, and for some of them a granulometric distribution.

Preferably, the sizes and size distributions of the particles of the compositions according to the invention are measured by static diffusion of light by means of a commercial granulometer of the MasterSizer 2000 type from Malvern. The data are processed on the basis of the Mie diffusion theory. This theory, precise for isotropic particles, makes it possible to determine an “effective” particle diameter in the case of non-spherical particles. This theory is described in particular in the book by Van de Hulst, H. C., “Light Scattering by Small Particles,” Chapters 9 and 10, Wiley, New York, 1957.

The composition is characterised by its “effective” mean diameter by volume D[4,3], defined as follows:

$$D [ 4 , 3 ] = \frac{{\sum }_{i} {V}_{i} \cdot {d}_{i}}{{\sum }_{i} {V}_{i}}$$

where Vᵢ represents the volume of the particles of effective diameter dᵢ. This parameter is in particular described in the technical documentation of the granulometer.

The measurements are made at 25° C., on a dilute dispersion of particles, obtained from the composition in the following manner: 1) dilution by a factor of 100 with water, 2) homogenisation of the solution, 3) resting of the solution for 18 hours, 4) recovery of the whitish, homogeneous supernatant.

The “effective” diameter is obtained taking a refractive index of 1.33 for water and a mean refractive index of 1.42 for the particles.

When it (they) is (are) present in the composition according to the invention, the content of additional wax(es) lies in the interval ranging from 0.5 to 15% by weight, better still from 1 to 10% by weight relative to the total weight of the composition.

The compositions according to the invention can contain one or more emulsifying surfactant agents, present in particular in a quantity ranging from 0.1 to 20%, and better from 0.3% to 15% by weight relative to the total weight of the composition.

According to the invention, an emulsifying surfactant agent appropriately selected for the obtention of an oil-in-water emulsion is generally used. In particular, an emulsifying surfactant agent having an HLB balance at 25° C. (hydrophile-lipophile balance) in the sense of GRIFFIN greater than or equal to 8 can be used.

The HLB value according to GRIFFIN is defined in J. Soc. Cosm. Chem. 1954 (volume 5), pages 249-256.

These surfactant agents can be selected from non-ionic, anionic, cationic and amphoteric surfactant agents. Reference can be made to the document “Encyclopedia of Chemical Technology, KIRK-OTHMER”, volume 22, p. 333-432, 3^{rd} edition, 1979, WILEY, for the definition of the properties and the emulsifying functions of the surfactant agents, in particular p. 347-377 of this reference, for the anionic, amphoteric and non-ionic surfactant agents.

The surfactant agents preferably used in the composition according to the invention are selected from:

a) non-ionic surfactants of HLB greater than or equal to 8 at 25° C., used alone or as a mixture, and the following can in particular be cited:

glycerol ethers, ethoxylated and/or propoxylated, which can contain from 1 to 150 ethylene oxide and/or propylene oxide units;

ethoxylated and/or propoxylated ethers (which can contain from 1 to 150 ethylene oxide and/or propylene oxide units) of fatty alcohols, in particular C₈-C₂₄, and preferably C₁₂-C₁₈, such as the ethoxylated ether of stearyl alcohol with 20 ethylene oxide units (CTFA name “Steareth-20”) such as the BRIJ 78 marketed by the company UNIQEMA, the ethoxylated ether of cetearyl alcohol with 30 ethylene oxide units (CTFA name “Ceteareth-30”) and the ethoxylated ether of the mixture of C₁₂-C₁₅ fatty alcohols containing 7 ethylene oxide units (CTFA name “C₁₂₋₁₅ Pareth-7”) such as that marketed under the name NEODOL 25-7® by SHELL CHEMICALS;

fatty acid esters, in particular C₈-C₂₄, and preferably C₁₆-C₂₂, and polyethylene glycol (or PEG) (which can contain from 1 to 150 ethylene oxide units), such as the PEG-50 stearate and PEG-40 monostearate marketed under the name MYRJ 52P® by the company UNIQEMA;

fatty acid esters, in particular C₈-C₂₄, and preferably C₁₆-C₂₂, and ethoxylated and/or propoxylated ethers of glycerol (which can contain from 1 to 150 ethylene oxide and/or propylene oxide units), such as the polyethoxylated glyceryl monostearate with 200 ethylene oxide units sold under the name Simulsol 220 TM® by the company SEPPIC; polyethoxylated glyceryl stearate with 30 ethylene oxide units such as the product TAGAT S® sold by the company GOLDSCHMIDT, polyethoxylated glyceryl oleate with 30 ethylene oxide units such as the product TAGAT O® sold by the company GOLDSCHMIDT, polyethoxylated glyceryl cocoate with 30 ethylene oxide units such as the product VARIONIC LI 13® sold by the company SHEREX, polyethoxylated glyceryl isostearate with 30 ethylene oxide units such as the product TAGAT L® sold by the company GOLDSCHMIDT and polyethoxylated glyceryl laurate with 30 ethylene oxide units such as the product TAGAT I® from the company GOLDSCHMIDT;

fatty acid esters, in particular C₈-C₂₄, and preferably C₁₆-C₂₂, and ethoxylated and/or propoxylated ethers of sorbitol (which can contain from 1 to 150 ethylene oxide and/or propylene oxide units), such as the polysorbate 60 sold under the name Tween 60® by the company UNIQEMA;

dimethicone copolyol, such as that sold under the name Q2-5220® by the company DOW CORNING;

dimethicone copolyol benzoate such as that sold under the name FINSOLV SLB 101® and 201® by the company FINTEX;

copolymers of propylene oxide and ethylene oxide, also called EO/PO polycondensates,

and mixtures thereof.

The EO/PO polycondensates are more particularly copolymers consisting of polyethylene glycol and polypropylene glycol blocks, such as for example polyethylene glycol/polypropylene glycol/polyethylene glycol triblock polycondensates. These triblock poly-condensates have for example the following chemical structure:

H—(O—CH₂—CH₂)ₐ—(O—CH(CH₃)—CH₂)_{b}—(O—CH₂—CH₂)ₐ—OH,

a formula wherein a ranges from 2 to 120, and b ranges from 1 to 100.

The EO/PO polycondensates preferably have a weight average molecular weight ranging from 1,000 to 15,000, and better ranging from 2,000 to 13,000. Advantageously, the EO/PO poly-condensates have a cloud point, at 10 g/l in distilled water, greater than or equal to 20° C., preferably greater than or equal to 60° C. The cloud point is measured in accordance with the standard ISO 1065. As EO/PO polycondensates utilisable according to the invention, the polyethylene glycol/polypropylene glycol/polyethylene glycol triblock polycondensates sold under the names SYNPERONIC® such as SYNPERONIC PE/L44® and SYNPERONIC PE/F127® by the company ICI can be cited.

b) non-ionic surfactant agents of HLB less than 8 to 25° C., which may be combined with one or more non-ionic surfactants of HLB greater than 8 to 25° C., such as those cited above, such as:

esters and ethers of sugars such as saccharose stearate, saccharose cocoate, sorbitan stearate and mixtures thereof, for example Arlatone 2121® marketed by the company ICI or SPAN 65V from the company UNIQEMA;

esters of fatty acids, in particular C₈-C₂₄, and preferably C₁₆-C₂₂, and polyols, in particular glycerol or sorbitol, such as glyceryl stearate, for example sold under the name TEGIN M® by the company GOLDSCHMIDT, glyceryl laurate such as the product sold under the name IMWITOR 312® by the company HULS, polyglyceryl-2 stearate, sorbitan tristearate, and glyceryl ricinoleate;

ethoxylated and/or propoxylated ethers such as the ethoxylated ether of stearyl alcohol with 2 ethylene oxide units (CTFA name “Steareth-2”) such as BRIJ 72 marketed by the company UNIQEMA;

the mixture of cyclomethicone/dimethicone copolyol sold under the name Q2-3225C® by the company DOW CORNING;

c) anionic surfactants such as:

salts of C₁₆-C₃₀ fatty acids, in particular amine salts such as triethanolamine stearate or 2-amino-2-methylpropane-1,3-diol stearate;

salts of polyethoxylated fatty acids, in particular amine salts or alkali metal salts, and mixtures thereof;

phosphate esters and salts thereof such as “DEA oleth-10 phosphate” (Crodafos N 10N from the company CRODA) or monopotassium monocetyl phosphate (Amphisol K from Givaudan or ARLATONE MAP 160K from the company UNIQEMA);

sulphosuccinates such as “Disodium PEG-5 citrate lauryl sulphosuccinate” and “Disodium ricinoleamido MEA sulphosuccinate”;

alkyl ether sulphates such as sodium lauryl ether sulphate;

isethionates;

acylglutamates such as “Disodium hydrogenated tallow glutamate” (AMISOFT HS-21 R® marketed by the company AJINOMOTO) and mixtures thereof.

As examples of cationic surfactants, the following can in particular be cited:
alkylimidazolidiniums such as isostearyl-ethylimidonium etho-sulphate,
ammonium salts such as (C₁₂₋₃₀ alkyl)-tri(C₁₋₄ alkyl)ammonium halides such as N,N,N-trimethyl-1-docosanaminium chloride (or Behentrimonium chloride).

The compositions according to the invention can also contain one or more amphoteric surfactants such as N-acyl amino acids such as N-alkyl-aminoacetates and disodium cocoamphodiacetate, and amine oxides such as stearamine oxide, or even silicone surfactants such as the dimethicone copolyol phosphates such as that sold under the name PECOSIL PS 100® by the company PHOENIX CHEMICAL.

According to one embodiment, the compositions according to the invention contain the following combination as the emulsifying system:
at least one C₁₀-C₃₀ alkyl phosphate surfactant agent, and
at least one ether of a C₈-C₂₄ fatty alcohol and polyethylene glycol, the ether containing from 1 to 19 ethylene oxide units and exhibiting an HLB <8 at 25° C.

According to one embodiment, the emulsifying system can further contain at least one ether of a C₈-C₂₄ fatty alcohol and polyethylene glycol, the ether containing from 20 to 1000 ethylene oxide units and being of HLB >8 to 25° C.

According to an advantageous embodiment, the cosmetic compositions according to the present invention contain less than 1%, preferably less than 0.5%, by weight of triethanolamine relative to the total weight of the composition, and better is free from triethanolamine.

According to an advantageous modification, the cosmetic compositions according to the invention contain less than 1% by weight, preferably less than 0.5% by weight, relative to the total weight of the composition, of triethanolamine stearate, and better is free from triethanolamine stearate

The compositions according to the invention can also contain at least one hydrophilic or lipophilic film-forming polymer.

In the sense of the present invention, “film-forming polymer” is understood to mean a polymer capable of forming, alone or in the presence of an auxiliary film-forming agent, a macroscopically continuous film adherent to the eyelashes, and preferably a cohesive film, and better still a film the cohesion and mechanical properties whereof are such that the film can be isolable and manipulable in isolation, for example when the film is created by pouring onto an anti-adhesive surface such as a teflon- or silicone-treated surface.

In general, when it (they) is or are present, the content of film-forming polymer(s) in the compositions according to the invention ranges from 0.1 to 40%, preferably from 0.5 to 30% and better from 1 to 20% by weight, relative to the total weight of the composition.

The hydrophilic film-forming polymer can be a water-soluble polymer or be in the form of a dispersion in an aqueous medium.

Among the film-forming polymers utilisable in the composition of the present invention, synthetic polymers of the radical type, or the polycondensate type, polymers of natural origin, and mixtures thereof can be cited.

As examples of water-soluble film-forming polymers, the following can be cited:

proteins such as proteins of plant origin such as wheat proteins and soya proteins, proteins of animal origin such as the keratins, for example keratin hydrolysates and keratin sulphonics;

polymers of cellulose such as hydroxyethylcellulose, hydroxy-propylcellulose, methylcellulose, ethylhydroxyethylcellulose, carboxy-methylcellulose, and quaternised derivatives of cellulose;

acrylic polymers or copolymers, such as polyacrylates or polymethacrylates;

vinyl polymers, such as polyvinylpyrrolidones, copolymers of methyl vinyl ether and maleic anhydride, copolymers of vinyl acetate and crotonic acid, copolymers of vinylpyrrolidone and vinyl acetate, copolymers of vinylpyrrolidone and caprolactam, and polyvinyl alcohols;

anionic, cationic, amphoteric or non-ionic polymers of chitin or chitosan;
gum arabics, guar gum, xanthan derivatives and gum karaya;
alginates and carrageenans;
glycoaminoglycans, hyaluronic acid and derivatives thereof;
shellac resin, gum sandarac, damars, elemis and copals;
desoxyribonucleic acid;
mucopolysaccharides such as the chondroitin sulphates, and mixtures thereof.

The film-forming copolymer can also be present in the composition in the form of particles dispersed in an aqueous phase, generally known under the name of latex or pseudolatex. The techniques for preparation of these dispersions are well known to the person skilled in the art.

As the aqueous dispersion of film-forming polymer, the acrylic dispersions sold under the names Neocryl XK-90®, Neocryl A-1070®, Neocryl A-1090®, Neocryl BT-62®, Neocryl A-1079® and Neocryl A-523® by the company AVECIA-NEORESINS, Dow Latex 432® by the company DOW CHEMICAL, Daitosol 5000 AD® or Daitosol 5000 SJ® by the company DAITO KASEY KOGYO, Syntran 5760® by the company Interpolymer Allianz, Opt® by the company Rohm and Haas; the aqueous dispersions of polyurethane sold under the names Neorez R-981® and Neorez R-974® by the company AVECIA-NEORESINS, Avalure UR-405®, Avalure UR-410®, Avalure UR-425®, Avalure UR-450®, Sancure 875®, Avalure UR-445® and Sancure 2060® by the company NOVEON, Impranil 85® by the company BAYER, Aquamere H-1511® by the company HYDROMER; the sulphopolyesters sold under the brand name Eastman AQ® by the company Eastman Chemical Products, vinyl dispersions such as Mexomère PAM®, aqueous dispersions of polyvinyl acetate such as “Vinybran®” from the company Nisshin Chemical or those marketed by the company UNION CARBIDE, aqueous dispersions of vinylpyrrolidone/dimethylamino-propyl methacrylamide/lauryldimethylpropylmethacrylamidoammonium chloride terpolymer such as Styleze W from ISP; aqueous dispersions of polyurethane/polyacrylic hybrid polymers such as those marketed under the names “Hybridur®” by the company AIR PRODUCTS or “Duromer®” from NATIONAL STARCH; “core/shell” type dispersions, for example those marketed by the company ATOFINA under the name Kynar (fluorinated core/acrylic shell) or else those described in the document U.S. Pat. No. 5,188,899 (silica core/silicone shell), and mixtures thereof can be used.

The lipophilic polymer can be in solution or dispersed in a non-aqueous solvent phase.

As examples of non-aqueous dispersions of film-forming polymer, the dispersions described, for example, in the document EP 749 746 and in particular particles of acrylic polymers, surface stabilised by a stabiliser, dispersed in a fatty phase (for example isododecane) such as Mexomere PAP® from the company CHIMEX, dispersions of particles of an ethylenic graft polymer, preferably acrylic, in a liquid fatty phase, the ethylenic polymer advantageously being dispersed in the absence of an additional surface stabiliser of particles such as described in particular in the document WO 04/055081 can be cited.

The compositions according to the present application can also contain at least one hydrophilic gelling agent. They can in particular be selected from:
homo- or copolymers of acrylic or methacrylic acids or salts thereof and esters thereof, and in particular the products sold under the names VERSICOL F® or VERSICOL K® by the company ALLIED COLLOID, UTRAHOLD 8® by the company CIBA-GEIGY, and polyacrylic acids of the SYNTHALEN K type,
copolymers of acrylic acid and acrylamide sold in the form of their sodium salt, under the names RETEN® by the company HERCULES, sodium salts of polyhydroxycarboxylic acids sold under the name HYDAGEN F® by the company HENKEL,
acrylic acid/alkyl acrylate copolymers of the PEMULEN type,
polymers of 2-acrylamido-2-methylpropanesulphonic acid (or AMPS) partially neutralised with ammonia and highly crosslinked, such as that marketed by the company CLARIANT,
AMPS/acrylamide copolymers of the SEPIGEL® or SIMULGEL® type marketed by the company SEPPIC, and
polyethoxylated AMPS/alkyl methacrylate copolymers, crosslinked or non-crosslinked, and mixtures thereof,
associative polyurethanes such as the C16-EO120-C16 polymer from the company SERVO DELDEN (marketed under the name SER AD FX1100, a molecule with a urethane function and weight average molecular weight of 1300), EO being an ethylene oxide unit, Rheolate 205 with a urea function sold by the company RHEOX or again Rheolate 208 or 204 (these polymers being sold in pure form) or DW 1206B from ROHM & HAAS with a C₂₀ alkyl chain and urethane linkage, sold with 20% active substance in water. Solutions or dispersions of these associative polyurethanes, in particular in water or in an aqueous alcohol medium, can also be used. As examples of such polymers, SER AD FX1010, SER AD FX1035 and SER AD 1070 from the company SERVO DELDEN and Rheolate 255, Rheolate 278 and Rheolate 244 sold by the company RHEOX can be cited. The product DW 1206F and DW 1206J, and Acrysol RM 184 Acrysol 44 from the company ROHM & HAAS, or else again Borchigel LW 44 from the company BORCHERS,
and mixtures thereof can also be used.

Certain water-soluble film-forming polymers cited above can also act as a water-soluble gelling agent.

When it (they) is (are) present in the composition according to the invention, the hydrophilic gelling agents can for example be present in the compositions according to the invention at a level ranging from 0.05 to 40% by weight relative to the total weight of the composition, preferably from 0.1 to 20% and better from 0.5 to 15% by weight.

The compositions according to the invention can also contain at least one colouring substance such as powdery substances, liposoluble colorants and water-soluble colorants.

The powdery colouring substances can be selected from the pigments and the pearl pigments.

The pigments can be white or coloured, mineral and/or organic, coated or uncoated. Among the mineral pigments, titanium dioxide, which may be surface treated, oxides of zirconium, zinc or cerium, and oxides of iron or chromium, manganese violet, ultramarine, chromium hydrate and Prussian blue can be cited. Among the organic pigments, carbon black, pigments of the D & C type, and lakes based on cochineal carmine, barium, strontium, calcium or aluminium can be cited.

The nacres can be selected from the white pearl pigments such as mica coated with titanium or bismuth oxychloride, coloured pearl pigments such as titanium mica with iron oxides, titanium mica with in particular Prussian blue or chromium oxide, titanium mica with an organic pigment of the aforesaid type and pearl pigments based on bismuth oxychloride.

The liposoluble dyes are for example Sudan red, D&C Red 17, D&C Green 6, β-carotene, soya oil, Sudan brown, D&C Yellow 11, D&C Violet 2, D&C Orange 5, quinoline yellow and annatto.

These colouring substances can be present at a level ranging from 0.01 to 30% by weight relative to the total weight of the composition.

The compositions according to the invention can also contain at least one filler.

The fillers can be selected from those well known to the person skilled in the art and currently used in cosmetic compositions. The fillers can be mineral or organic, lamellar or spherical. Talc, mica, silica, kaolin, polyamide powders such as Nylon® marketed under the name Orgasol® by the company Atochem, poly-β-alanine and polyethylene, tetrafluoroethylene polymer powders such as Teflon®, lauroyl-lysine, starch, boron nitride, expanded polymeric microspheres such as those of polyvinylidene chloride/acrylonitrile such as those marketed under the name Expancel® by the company Nobel Industrie, acrylic powders such as those marketed under the name Polytrap® by the company Dow Corning, polymethyl methacrylate particles and silicone resin microbeads (Tospearls® from Toshiba, for example), precipitated calcium carbonate, magnesium carbonate and hydro-carbonate, hydroxyapatite, hollow silica microspheres (Silica Beads® from MAPRECOS), microcapsules of glass or ceramic, metal soaps derived from organic carboxylic acids having from 8 to 22 carbon atoms, and in particular from 12 to 18 carbon atoms, for example zinc, magnesium or lithium stearate, zinc laurate and magnesium myristate can be cited.

A compound capable of swelling on heating and in particular thermo-expandable particles such as non-expanded microspheres of vinylidene chloride/acrylonitrile/methyl methacrylate copolymer or acrylonitrile homopolymer such as for example those marketed respectively under the names Expancel® 820 DU 40 and Expancel® 007WU by the company AKZO NOBEL can also be used.

The fillers can represent from 0.1 to 25%, in particular from 0.2 to 20% by weight relative to the total weight of the composition.

The compositions according to the invention can also contain at least one fibre which enables improvement of the lengthening effect.

“Fibre” should be understood to mean an object of length L and diameter D such that L is much greater than D, D being the diameter of the circle in which the cross-section of the fibre is inscribed. In particular, the L/D ratio (or form factor) is selected in the band ranging from 3.5 to 2500, in particular from 5 to 500, and more particularly from 5 to 150.

The fibres utilisable in the composition of the invention can be fibres of synthetic or natural origin, mineral or organic. They can be short or long, unitary or structured, for example, braided, hollow or full. They can be of any shape and in particular of circular or polygonal cross-section (square, hexagonal or octagonal) depending on the specific application envisaged. In particular, their ends are blunted and/or polished to avoid injury.

In particular, the fibres have a length ranging from 1 μm to 10 mm, preferably from 0.1 mm to 5 mm, and more particularly from 0.3 mm to 3.5 mm. Their cross-section can be contained in a circle of diameter ranging from 2 nm to 500 μm, in particular ranging from 100 nm to 100 μm and more particularly from 1 μm to 50 μm. The weight or titre of the fibres is often given in denier or decitex and represents the weight in grams for 9 km of yarn. The fibres utilisable in the invention can in particular have a titre selected in the band ranging from 0.15 to 30 denier and in particular from 0.18 to 18 denier.

The fibres utilisable in the composition of the invention can be selected from rigid or non-rigid fibres. They can be of synthetic or natural origin, mineral or organic.

Moreover, the fibres can be surface treated or not, coated or not, and coloured or not.

As fibres utilisable in the composition according to the invention, non-rigid fibres such as polyamide fibres (Nylon®) or rigid fibres such as polyimide-amide fibres such as those sold under the names KERMEL®, KERMEL TECH® by the company RHODIA, or poly-(p-phenylene-terephthalamide) (or aramide) fibres in particular sold under the name Kevlar® by the company DUPONT DE NEMOURS can be cited.

The fibres can be present in the composition according to the invention at a level ranging from 0.01% to 10% by weight, relative to the total weight of the composition, in particular from 0.1% to 5% by weight, and more particularly from 0.3% to 3% by weight.

The compositions according to the invention can also further contain at least one cosmetic active substance.

As cosmetic active substances which can be used in the compositions according to the invention, antioxidants, preservatives, perfumes, neutralising agents, emollients, thickeners, coalescing agents, plasticisers, moisturisers, vitamins and filters, in particular sun filters and mixtures thereof can in particular be cited.

The person skilled in the art will of course take care to choose any complementary additives and/or the quantity thereof in such a manner that the advantageous properties of the composition according to the invention are not, or essentially not, impaired by the addition envisaged.

Preferably the composition according to the invention is not rinsed.

The composition according to the invention can be packed in a container defining at least one compartment which contains the composition, the container being closed by a closure element.

The container is preferably combined with an applicator, in particular in the form of a brush comprising an arrangement of bristles held in position by a twisted wire. Such a twisted brush is in particular described in the U.S. Pat. No. 4,887,622. It can also be in the form of a comb comprising a plurality of application components, obtained in particular by moulding. Such combs are for example described in the patent FR 2,796,529. The applicator can be integral with the container, as for example described in the patent FR 2,761,959. Advantageously, the applicator is integral with a rod which is itself integral with the closure element.

The closure element can be attached to the container by screwing on. Alternatively, the attachment between the closure element and the container is effected other than by screwing on, in particular via a bayonet mechanism, by a ratchet mechanism or by clamping. “Ratchet mechanism” is in particular understood to mean any system involving the crossing of a rim or a band of material by elastic deformation of one portion, in particular of the closure element, then by return of the portion to a position not elastically constrained after the cross of the rim or the band.

The container can at least in part be made of thermoplastic material. As examples of thermoplastic materials, polypropylene or polyethylene can be cited.

Alternatively, the container is made of a non-thermoplastic material, in particular of glass or of metal or alloy.

The container is preferably equipped with a wiper located in the vicinity of the opening of the container. Such a wiper makes it possible to wipe the applicator and possibly the rod whereof it can be an integral part. Such a wiper is for example described in the patent FR 2,792,618.

The compositions according to the invention can be used for making up keratinous fibres, in particular the eyelashes, for example as mascaras.

Another subject matter of the invention is a process for cosmetic treatment of keratinous fibres, such as the eyelashes, and more particularly for making up or coating the fibres, which consists in applying an effective quantity of a composition such as described above onto the fibres.

The following examples illustrate the present invention. The quantities indicated below are expressed in percentage by weight relative to the total weight of the composition.

### EXAMPLES

### Example 1

A mascara was prepared from the ingredients shown in the table below.

| Ingredients | |
|---|---|
| Candelilla wax | 16 |
| Carnauba wax | 4 |
| Trans isomerised jojoba oil (Simmondsia Chinensis) | 5 |
| of M. Pt. 45° C. ⁽¹⁾ | |
| Black iron oxide | 7 |
| Gum arabic | 3.4 |
| Hydroxyethylcellulose | 0.9 |
| Potassium cetyl phosphate ⁽²⁾ | 2.5 |
| Ethoxylated stearyl alcohol (20 ethylene oxide or | 5.2 |
| (EO) units) ⁽³⁾ | |
| Ethoxylated stearyl alcohol (2 EO units) ⁽⁴⁾ | 2.4 |
| Pure cetyl alcohol | 2.3 |
| Preservatives | qs |
| Water | qsp 100 |

⁽¹⁾ sold under the brand name Iso-Jojoba-50 ® by the company Desert Whale
⁽²⁾ sold under the brand name Amphisol K by the company Givaudan
⁽³⁾ sold under the brand name BRIJ 78 by the company UNIQEMA
⁽⁴⁾ sold under the brand name BRIJ 72 by the company UNIQEMA

This mascara composition has a content of hard waxes of 22.3% by weight, a content of pasty compounds of 5% by weight and a hard waxes/(pasty compounds) weight ratio of 4.46.

The ingredients of the fatty phase were mixed, and it was heated to 98° C. The aqueous phase, previously heated to 93° C., was then added with stirring.

A texture particularly suitable for application by brush was obtained, which resulted in a thick making up of the eyelashes.

### Example 2

A mascara was prepared from the ingredients shown in the table below.

| Ingredients | |
|---|---|
| Candelilla wax | 7.86 |
| Carnauba wax | 3.21 |
| Trans isomerised jojoba oil (Simmondsia Chinensis) | 4.07 |
| of M. Pt. 45° C. ⁽¹⁾ | |
| Shea butter ⁽²⁾ | 5 |
| Black iron oxide | 7.14 |
| Gum arabic | 3.4 |
| Hydroxyethylcellulose | 0.9 |
| Potassium hexadecyl-hydrogenophosphate ⁽³⁾ | 2.18 |
| Ethoxylated stearyl alcohol (20 EO units) ⁽⁴⁾ | 4.44 |
| Ethoxylated stearyl alcohol (2 EO units) ⁽⁵⁾ | 2.1 |
| Pure cetyl alcohol | 2 |
| Simethicone | 0.13 |
| Preservatives | qs |
| Water | qsp 100 |

⁽¹⁾ sold under the brand name Iso-Jojoba-50 ® by the company Desert Whale
⁽²⁾ sold under the brand name Lipex Sheasoft by the company AARHUSKARLSHAMN
⁽³⁾ sold under the brand name Arlatone MAP 160K by the company UNIQEMA
⁽⁴⁾ sold under the brand name BRIJ 78 by the company UNIQEMA
⁽⁵⁾ sold under the brand name BRIJ 72 by the company UNIQEMA

This mascara composition has a content of hard waxes of 13.07% by weight, a content of pasty compounds of 9.07% by weight and a hard waxes/(pasty compounds) weight ratio of 1.44.

### Example 3

A mascara was prepared from the ingredients shown in the table below.

| Ingredients | |
|---|---|
| Candelilla wax | 7.86 |
| Carnauba wax | 3.21 |
| Trans isomerised jojoba oil (Simmondsia Chinensis) | 4.07 |
| of M. Pt. 45° C. ⁽¹⁾ | |
| Orange wax ⁽²⁾ | 5 |
| Black iron oxide | 7.14 |
| Gum arabic | 3.4 |
| Hydroxyethylcellulose | 0.9 |
| Potassium hexadecyl-hydrogenophosphate ⁽³⁾ | 2.18 |
| Ethoxylated stearyl alcohol (20 EO units) ⁽⁴⁾ | 4.44 |
| Ethoxylated stearyl alcohol (2 EO units) ⁽⁵⁾ | 2.1 |
| Pure cetyl alcohol | 2 |
| Simethicone | 0.13 |
| Preservatives | qs |
| Water | qsp 100 |

⁽¹⁾ sold under the brand name Iso-Jojoba-50 ® by the company Desert Whale
⁽²⁾ sold under the brand name Orange Peel Wax by the company Koster Keunen
⁽³⁾ sold under the brand name Arlatone MAP 160K by the company UNIQEMA
⁽⁴⁾ sold under the brand name BRIJ 78 by the company UNIQEMA
⁽⁵⁾ sold under the brand name BRIJ 72 by the company UNIQEMA

This mascara composition has a content of hard waxes of 13.07% by weight, a content of pasty compounds of 9.07% by weight and a hard waxes/(pasty compounds) weight ratio of 1.44.

The above written description of the invention provides a manner and process of making and using it such that any person skilled in this art is enabled to make and use the same, this enablement being provided in particular for the subject matter of the appended claims, which make up a part of the original description and include a composition comprising an aqueous phase, at least 10% by weight of at least one hard wax, relative to the total weight of the composition, and at least one pasty compound.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

What is claimed is:

1. A composition comprising an aqueous phase, at least 10% by weight of at least one hard wax relative to the total weight of the composition, and at least one pasty compound.

2. The composition according to claim 1, further comprising at least one oil.

3. The composition according to claim 1, wherein the hard wax(es)/(pasty compound(s)) weight ratio is 2 to 8.

4. The composition according to claim 2, wherein the hard wax(es)/(pasty compound(s)+possible oil(s)) weight ratio is 2 to 8.

5. The composition according to any one of claim 2, wherein the total quantity of pasty compound(s) and oil(s) is greater than 1% by weight relative to the total weight of the composition.

6. The composition according to claim 1, wherein the hard wax or waxes exhibit a hardness at 20° C. greater than 5 MPa.

7. The composition according to claim 1, wherein the hard wax or waxes is/are selected from the group consisting of Carnauba wax, candelilla wax, the wax bis-PEG-12 dimethicone candelillate, hydrogenated Jojoba wax, hydrogenated palm oil, rice bran wax, Sumac wax, ceresin waxes, laurel wax, Chinese insect wax, Shellac wax, hydrogenated olive oil, waxes obtained by hydrogenation of olive oil esterified with C12 to C18 chain fatty alcohols, waxes obtained by hydrogenation of castor oil esterified with cetyl or behenyl alcohol, hydrogenated Cameline wax, Ouricury wax, Montan wax, ozokerite waxes, microcrystalline waxes, triglycerides of lauric, palmitic, cetylic and stearic acids, polymethylene waxes, polyethylene waxes, alcohol-polyethylene waxes, 95/5 ethylene/acrylic acid copolymer, hydroxyoctacosanyl hydroxystearate, octacosanyl stearate, stearyl stearate, pentaerythritol distearate, the mixture of dibehenyl adipate, dioctadecyl adipate and di-eicosanyl adipate, the mixture of dilauryl adipate and ditetradecyl adipate, the mixture of dioctadecyl sebacate, didocosyl sebacate and dieicosyl sebacate, the mixture of dioctadecyl octadecanedioate, didocosyl octanedioate and dieicosyl octanedioate, pentaerythrityl tetrastearate, tetracontanyl stearate, stearyl benzoate, behenyl fumarate, di-(trimethylol-1,1,1-propane) tetrastearate, didotriacontanyl distearate, the montanate of polyethylene glycol with 4 ethylene oxide units, hexanediol disalicylate, dipentaerythritol hexastearate, ditrimethylolpropane tetrabehenate, Jojoba esters, mixtures of linear (C20-40) carboxylic acid/saturated hydrocarbons, synthetic wax of the Fischer-Tropsch type, cetyl alcohol, stearyl alcohol, behenyl alcohol, dioctadecyl carbonate, saccharose polybehenate, and mixtures thereof.

8. The composition according to claim 7, wherein the hard wax or waxes is/are selected from the group consisting of carnauba wax, candelilla wax, hydrogenated jojoba wax, sumac wax, waxes obtained by hydrogenation of olive oil esterified with C12 to C18 chain fatty alcohols, rice bran wax, cetyl, stearyl and behenyl alcohols, laurel wax, Ouricury wax, and mixtures thereof.

9. The composition according to claim 1, wherein the composition contains at least 12% by weight of at least one hard wax relative to the total weight of the composition.

10. The composition according to claim 1, wherein the pasty compound or compounds are lipophilic fatty compounds with a reversible solid/liquid change of state and having a liquid fraction and a solid fraction at a temperature of 23° C.

11. The composition according to claim 10, wherein the liquid fraction of the pasty compound, measured at 23° C., represents from 20 to 97% by weight of the pasty compound.

12. The composition according to claim 1, wherein the pasty compound or compounds are selected from synthetic compounds and compounds of plant origin.

13. The composition according to claim 12, wherein the pasty compound or compounds are selected from the group consisting of:
lanolin and derivatives thereof,
polymeric or non-polymeric silicone compounds,
polymeric or non-polymeric fluorinated compounds,
vinyl polymers, including
homopolymers of olefins,
copolymers of olefins,
homopolymers and copolymers of hydrogenated dienes,
linear or branched, homo or copolymeric oligomers of alkyl (meth)acrylates preferably having a C₈-C₃₀ alkyl group
homo and copolymeric oligomers of vinyl esters having C₈-C₃₀ alkyl groups,
homo and copolymeric oligomers of vinyl ethers having C₈-C₃₀ alkyl groups,
liposoluble polyethers resulting from polyetherification between one or more C₂-C₁₀₀ diols,
esters and polyesters,
and mixtures thereof.

14. The composition according to claim 12, wherein the pasty compound is selected from trans isomerised partially hydrogenated jojoba oil, orange wax, shea butter, partially hydrogenated olive oil, cocoa butter, mango oil and mixtures thereof.

15. The composition according to claim 1, wherein the pasty compound or compounds are present in a quantity greater than 1% by weight relative to the total weight of the composition.

16. The composition according to claim 2, wherein the oil or oils are volatile or non-volatile, and are selected from hydrocarbon oils, silicone oils, fluorinated oils and mixtures thereof.

17. The composition according to claim 2, wherein the oil is present in a quantity ranging from 0.01 to 30% by weight relative to the total weight of the composition.

18. The composition according to claim 1, wherein it exhibits a dry matter content greater than or equal to 40% by weight relative to the total weight of the composition.

19. The composition according to claim 1, wherein the aqueous phase comprises water and at least one water-soluble solvent.

20. The composition according to claim 19, wherein the water-soluble solvent or solvents is/are selected from the group consisting of lower monohydric alcohols having from 1 to 5 carbon atoms, alkylene glycols having from 2 to 8 carbon atoms, C₃-C₄ ketones, C₂-C₄ aldehydes and mixtures thereof.

21. The composition according to claim 1, wherein the aqueous phase is present in an amount of 1 to 95% by weight relative to the total weight of the composition.

22. The composition according to claim 1, further comprising at least one additional wax exhibiting a hardness less than or equal to 5 MPa at 20° C.

23. The composition according to claim 22, wherein the additional wax or waxes are selected from the group consisting of beeswaxes, siliconised beeswax, paraffin waxes, berry wax, lemon wax, 85/15 ethylene/vinyl acetate copolymer, silicone wax and mixtures thereof.

24. The composition according to claim 22, wherein the additional wax or waxes are adhesive waxes exhibiting an adhesivity greater than or equal to 0.1 N·s and a hardness less than or equal to 3.5 MPa.

25. The composition according to claim 24, wherein the adhesive wax or waxes are selected from the C₁₈-C₃₈ alkyl (hydroxystearoyl)stearates.

26. The composition according to claim 22, wherein the additional wax or waxes are present in an amount of 0.5 to 15% by weight relative to the total weight of the composition.

27. The composition according to claim 1, further comprising one or more emulsifying surfactant agents.

28. The composition according to claim 27, wherein the emulsifying surfactant agent or agents are present in an amount of 0.1 to 20% by weight relative to the total weight of the composition.

29. The composition according to claim 27, comprising the following combination:
at least one C₁₀-C₃₀ alkyl phosphate surfactant agent, and
at least one C₈-C₂₄ fatty alcohol ether and polyethylene glycol, the ether having from 1 to 19 ethylene oxide units and exhibiting an HLB <8 to 25° C.

30. The composition according to claim 1, comprising less than 1%, by weight of triethanolamine relative to the total weight of the composition.

31. The composition according to claim 30, wherein it is free of triethanolamine.

32. The composition according to claim 1, comprising less than 1% by weight of triethanolamine stearate, relative to the total weight of the composition.

33. The composition according to claim 32, wherein it is free from triethanolamine stearate.

34. The composition according to claim 1, further comprising at least one hydrophilic or lipophilic film-forming polymer.

35. The composition according to claim 1, further comprising at least one hydrophilic gelling agent.

36. The composition according to claim 1, further comprising at least one fiber.

37. A process for making up a keratinous fiber, comprising applying a quantity of the composition according to claim 1 to the fiber.
